(19) United States
(12) Patent Application Publication
(10) Pub. No.: US 2011/0229446 A1
(43) Pub. Date: Sep. 22, 2011

(54) Method and composition to relieve sexual discomfort and improve vaginal health

(76) Inventors: Stephen B. Roman, Phoenix, AZ (US); Gary S. Kehoe, Glendale, AZ (US)

(21) Appl. No.: 12/932,537

(22) Filed: Feb. 28, 2011

Related U.S. Application Data
(60) Provisional application No. 61/339,343, filed on Mar. 2, 2010.

(51) Int. Cl. A61K 35/74; A61K 31/715; A61P 15/00

(52) U.S. Cl. 424/93.45; 514/54

(57) ABSTRACT

A method to reduce sexual discomfort for a woman during sexual intercourse vaginally introduces a lubricant composition comprising carrageenan comprising 0 to 92% by weight iota carrageenan, 10% to 100% by weight lambda carrageenan, and, 0 to 18% by weight kappa carrageenan; water such that the ratio of carrageenan to water is in the range of 1:10 to 1:50; at least one prebiotic; and, a natural humectant with a peak tackiness force of less than 50 g and a work of adhesion of less than 0.500 GS.

This application claims priority based on provisional patent application Ser. No. 61/339,343, filed Mar. 2, 2010.

This invention relates to vaginally applied compositions.

More particularly, the invention relates to personal lubricants and to compositions to improve vaginal health.

Research and development in connection with producing improved vaginally applied compositions has been ongoing for many years in connection with personal lubricants and compositions to facilitate and improve vaginal health.

Accordingly, it would be highly desirable to produce improved vaginal compositions.

Therefore, it is a principal object of the invention to provide improved vaginal compositions.

This, and other further more specific objects and advantages of the invention will be apparent from the following detailed description thereof.

We have discovered a personal lubricant composition which, while having a reduced coefficient of friction, improves the health and increases the population of vaginal microflora. The composition includes a lubricant, a probiotic, and a natural humectant. The composition can also include one or more ingredients selected from a group comprising prebiotics, vasodilators, odorants, topical sensates, moisturizier, and soft tissue soothing agents, a pH adjuster, and a preservative. The composition has a viscosity and pH which permit the composition to be administered in the vaginal canal in humans.

The preferred pH for each of the compositions described herein is in the range of 3.2 to 7.0, preferably 3.8 to 6.5, most preferably 4.0 to 5.5.

The preferred viscosity for each of the composition described herein is in the range of 200 to 50,000 cps, preferably 1,000 to 20,000 cps, most preferably 2,000 to 18,000 cps.

In another embodiment of the invention, a lubricant is combined with one or more ingredients selected from a group comprising probiotics, prebiotics, propanediol, vasodilators, odorants, topical sensates, moisturizers, soft tissue soothing agents, citric acid and a preservative.

In a further embodiment of the invention, a lubricant is combined with one or more ingredients selected from a group comprising probiotics, prebiotics, natural propanediol, vasodilators, odorants, topical sensates, moisturizers, soft tissue soothing agents, a pH adjuster, and a preservative. As used herein a natural propanediol is a propanediol derived from corn or another plant and is not a propanediol that is derives from petroleum or some other source.

In still another embodiment of the invention, a lubricant is combined with one or more ingredients selected from a group comprising the probiotic Ganaden BC30 (Bacillus coagulans), the prebiotic agave inulin spray dry, the humectant 1,3 propanediol from maize, the vasodilator niacin, the topical sensate menthol, the soft tissue soothing agent aloe vera or vitamin E, the pH adjuster citric acid, and a preservative. The preservative is comprised of one or more selected from a group consisting of sodium benzoate, potassium sorbate, or sodium hydroxymethylglycinate. Spray dry agave inulin is more soluble than liquid versions of inulin and does not contain the high levels of sugar found in liquid versions of inlin.

In still a further embodiment of the invention, carrageenan hydrated in water and 1,3 propanediol from maize are combined with one or more ingredients selected from a group comprising the probiotic Ganaden BC30 (Bacillus coagulans), the prebiotic agave inulin spray dry, a soft tissue soothing agent such as aloe vera or vitamin E, a pH adjuster such as citric acid, and a preservative. The preservative is comprised of one or more selected from a group consisting of sodium benzoate, potassium sorbate, or sodium hydroxymethylglycinate.

In yet still another embodiment of the invention, a first liquid composition comprising a lubricant such as carrageenan hydrated in water and a humectant such as propanediol, and a prebiotic such as inuline and a second liquid composition containing a probiotic such a Genaden BC30 are applied simultaneously to the vaginal area using a dual-chambered, airless dispenser which houses each liquid in a chamber separate from the other. When the dispenser nozzle is manually actuated, liquid from each chamber passes into the nozzle, intermixes with liquid from the other chamber and is dispensed from the dispenser. In an alternate embodiment of the invention, the first liquid passes through a first conduit in the nozzle and the second liquid passes through a second conduit separate from the first so that the liquids contact each other only after they have been dispensed from the dispenser.

The weight percents of various ingredients which can, if desired, be utilized in the composition of the invention are (when utilized) (1) lubricant (such as carrageenan) 0.1% to 10% by weight, preferably 0.75 to 3.5% by weight; (2) water 65% to 99.9% by weight, preferably 70% to 80% by weight, (3) humectant (such a 1,3 propanediol), 0.001% to 100% by weight, preferably 0.1% to 80% by weight, most preferably 0.5% to 15% by weight, (4) probiotic (scy as Ganaden BC30) 0.001% to 3.0% by weight, preferably 0.01% to 1.0% by weight, (5) prebiotic (such as agave inulin spray dry) 0.001% to 5.0% by weight, preferably 0.1% to 0.5% by weight, (6) soft tissue soother (such as aloe vera or Vitamin E) 0.01% to 20% by weight, preferably 0.1 to 0.5% by weight, (7) aloe vera as soft tissue soother 0.01% to 20% by weight, preferably 0.1% to 0.5% by weight, (8) Vitamin E as soft tissue soother 0.01% to 5.0% by weight, preferably 0.1% to 1.0% by weight, (9) vasodilator (such as L-arginine or niacin) 0.001% to 5.0% by weight, preferably 0.05 to 1.0% by weight, (10) L-arginine as vasodilator 0.001% to 5.0% by weight, preferably 0.05% to 1.0% by weight, (11) niacin as vasodilator 0.001% to 2.5% bye weight, preferably 0.05 to 1.0% by weight, (12) pH adjuster (such as citric acid) 0.01% to 5.0% by weight, preferably 0.1% to 0.8%% by weight, (13) topical sensate (such as menthol or primrose oil) 0.001% to 1.5% by weight, preferably 0.01% to 0.25% by weight, (14) menthol as a topical sensate 0.001% to 1.0% by weight, preferably 0.01% to 0.1% by weight, (15) evening primrose oil as a sensate 0.01% to 2.0% by weight, preferably 0.1% to 1.0% by weight, and (16) preservative (such as sodium hydroxymethlyglycinate, propylparaben or methylparaben) 0.01% to 1.0% by weight, preferably 0.25 to 0.75% by weight.

Examples of lubricants that may be used are silicone, dimethicone, ethylmethylcellulose, methylcellulose, carrageenan, glycerine, polyhidritic glycerols, carboxymethylcellulose, guar gums, alginates, and propylene glycol. Proprietary combinations of iota, lambda, and kappa carrageenan can, as discussed below, be utilized.

Examples of preservatives that may be used are methyl paraben, propyl paraben, sorbate, and sodium hydroxymethylglycinate.

Examples of probiotics that may be used are Ganaden BC30, Lactobacillus, and Bifodobacteria. As used herein a probiotics is a microorganism thought to be healthy for the host organism.

Examples of prebiotics that may be used are fructooligosaccharides, and inulin from chicory or agave. As used herein, a prebiotic is a non-digestible food ingredient that stimulates the growth and/or activity of bacteria in the digestive system which are beneficial to the health of the body.

Examples of vasodilators that may be used are arginine, niacin, nephridine, aminophylline, peppermint oil. As used herein, a vasodilator is a composition that functions to dilate blood vessels.

Examples of odorants that may be used are lavender oil, clove oil, musk oil, rose oil, sandalwood oil, jasmine oil, cinnamon oil, nutmeg oil, evening primrose oil, rosemary oil. As used herein an odorant is a topical ingredient that produces a perceptible odor.

Examples of topical sensates that may be used are menthol compositions in solid crystals or liquid forms, evening primrose oil, cinnamon oil, peppermint oil, rosemary oil, and sweet birch oil. A sensate is something perceived by the senses. More particularly, as used herein a sensate is a topical ingredient that induces a sensation of cooling or warming.

Examples of moisturizers (humectants) that may be used are 1,3 propanediol from maize, 1,3 propanediol from petroleum sources, polyhydritic alcohols, and glycerin. As used herein, a moisturizer is a topical ingredient that increases the softness and suppleness of skin by penetrating into skin tissue.

Examples of soft tissue soothing agents that may be used are vitamin E, and aloe vera compositions in the form of oils, concentrates, or water soluble gels. As used herein, a soft tissue soothing agent is a topical ingredient that repairs and heals epithelial layer cells.

Examples of pH adjusters that may be used are citric acid, hydrogen peroxide, lactic acid, and sodium hydroxide.

### EXAMPLE I

The following ingredients are provided in quantities to be admixed to produce a final composition having the ingredients in the weight percent noted:

| | Weight % in |
|---|---|
| Ingredient | final composition |
| Carrageenan | 2.0 |
| Water | QS |
| Propanediol (Zemea TM) | 8.0 |
| Ganaden BC30 | 0.01% |
| Agave Inulin, spray dry | 0.50% |
| Menthol | 0.15% |
| Niacin | 0.05% |
| L-Arginine | 0.10% |
| Citric Acid | 0.13% |
| Evening Primrose Oil | 1.0% |
| Potassium Sorbate | 0.05% |
| Sodium Benzoate | 0.05% |
| Sodium Hydroxymethylglycinate | 0.1% |

### Mixing Procedure

The water is metered into a first mixing vessel, and agitation is engaged.

The potassium sorbate, sodium benzoate, sodium hydroxymethylglycinate, citric acid, inulin, Ganaden BC30, evening primrose oil, menthol, niacin and L-arginine are added to the water an mixed to uniformity.

In a second mixing vessel the propanediol and carrageenan are mixed together to uniformity and are then admixed to the composition in the first mixing vessel. At his time further additives, medicaments, ingredients, etc. may be added and blended to uniformity.

The process can be carried out by a batch process, a continuous process or a combination of the two. The resulting lubricant composition may be scented or unscented, colored or colorless, flavored or flavorless, water-based, and/or may include humectants. The vaginal formula is pH balance with a range between 3.2 and 10.6, currently preferably 4.0. The viscosity range is between 200 to 30,000 cps, currently preferably 2,500 cps.

### EXAMPLE II

The following ingredients are provided in quantities to be admixed to produce a final lubricant composition having the ingredients in the weight percent noted:

| Ingredient | Weight % in final composition |
|---|---|
| Carrageenan (90.5% by weight Lambda, | 2.0 |
| 5.7% by weight Iota; 3.8% by weight | |
| Kappa) | |
| Water | QS |
| Propanediol (Zemea TM) | 8.0 |
| Ganaden BC30 | 0.01% |
| Agave Inulin, spray dry | 0.50% |
| Menthol | 0.15% |
| Niacin | 0.05% |
| L-Arginine | 0.10% |
| Citric Acid | 0.13% |
| Evening Primrose Oil | 1.0% |
| Potassium Sorbate | 0.05% |
| Sodium Benzoate | 0.05% |
| Sodium Hydroxymethylglycinate | 0.1% |

### Mixing Procedure

The water is metered into a first mixing vessel, and agitation is engaged.

The potassium sorbate, sodium benzoate, sodium hydroxymethylglycinate, citric acid, inulin, Ganaden BC30, evening primrose oil, menthol, niacin and L-arginine are added to the water an mixed to uniformity.

In a second mixing vessel the propanediol and carrageenan are mixed together to uniformity and are then admixed to the composition in the first mixing vessel. At his time further additives, medicaments, ingredients, etc. may be added and blended to uniformity.

The process can be carried out by a batch process, a continuous process or a combination of the two. The resulting lubricant composition may be scented or unscented, colored or colorless, flavored or flavorless, water-based, and/or may include humectants. The vaginal formula is pH balanced with a range between 3.2 and 10.6, currently preferably 4.0. The viscosity range is between 200 to 30,000 cps, currently preferably 2,500 cps. The peak tackiness force is less than 50.0 g and the work of adhesion (area) (GS) is less than 0.500.

The carrageenan composition in the above noted lubricant formulation includes 0 to 92%, preferably 1.0% to 90% by weight iota; 1 to 100%, preferably 10% to 100% by weight lambda; and, 0 to 18%, preferably 0.001 to 15% by weight kappa carrageenan. This carrageenan mixture is important because it facilitates controlling the viscosity of the composition while also facilitating balancing the lubricity and tackiness of the formulation. In one preferred embodiment of the invention, the carrageenan composition includes at least 85% by weight lambda carrageenan, less than 15% by weight iota carrageenan, and less than 10% by weight kappa carrageenan.

When carrageenan is utilized in a formulation that is not a lubricant but is primarily intended to be used as a moisturizer and as a healing composition, the carrageenan composition includes 10% to 100% by weight iota carrageenan, 0% to 30% kappa carrageenan, and 0 to 90% lambda carrageenan.

The ratio of carrageenan to water is important in preparing lubricant compositions in accordance with the invention. If insufficient water is present lubricity is adversely affected, as is the effectiveness of some humetants. The ratio of carrageenan to water is in the range of 1:10 to 1:50, preferably 1:20 to 1:48, most preferably 1:30 to 1:45. Insufficient water can cause a lubricant composition to dry prematurely.

The composition also specifically utilizes the natural humectant 1,3 propanediol because it produces a composition having low tack. As used herein, tack indicates the stickiness of a composition. One company which produces equipment to measure the tackiness of gels is Texture Technologies Corp of 18 Fairview Road, Scarsdale, New York10583 914 472 0531, sales@texturetechnologies.com. The peak tackiness force of a lubricant produced in accordance with the invention is less than 50.0 g, and the work of adhesion is less than 500 (area)(GS).

### EXAMPLE III

A one gram sample of the lubricant formulation prepared in accordance with Example II is placed on a horizontally oriented Lexan plate and a probe is downwardly displaced onto the sample at a speed of one mm/sec until a downward compressive force of 500 g is developed and held for one second, after which the probe is withdrawn at a speed of 10 mm/sec for a distance of 5 mm from the Lexan plate. The probe comprises a 3 inch diameter acrylic cylinder plate. The adhesive force produced by the lubricant formulation is measured when the probe is 5 mm. This measurement indicates that the peak tackiness force of the lubricant formulation is less than 50.0 grams and that the work of adhesion is less than 500 (area)(GS). Although any desired equipment can be utilized to carry out this measurement of peak tackiness force and the work of adhesion, one piece of equipment which can be utilized to accomplish the foregoing and measure peak tackiness force and the work of adhesion is the TA.XT2 model with 25 kg capacity in combination with a TA-90 platform. This equipment is manufactured by the above noted Texture Technologies Corp of 18 Fairview Road, Scarsdale, N.Y. 10583 (914) 472-0531.

### EXAMPLE IV

The following ingredients are provided in quantities to be admixed to produce a final vaginal moisturizer/healing composition having the ingredients in the weight percent noted:

| | Weight % in |
|---|---|
| Ingredient | final composition |
| Carrageenan | 3.0 |
| Water | QS |
| Propanediol (Zemea TM) | 15.0 |
| Ganaden BC30 | 0.01% |
| Agave Inulin, spray dry | 0.50% |
| Menthol | 0.15% |
| Citric Acid | 0.13% |
| Potassium Sorbate | 0.05% |
| Sodium Benzoate | 0.45% |
| Sodium Hydroxymethylglycinate | 0.1% |
| Aloe | 0.1% |
| Vitamin E | 0.1% |

### Mixing Procedure

The water is metered into a first mixing vessel, and agitation is engaged.

The potassium sorbate, sodium benzoate, sodium hydroxymethylglycinate, citric acid, inulin, Ganaden BC30, evening primrose oil, menthol, niacin and L-arginine are added to the water an mixed to uniformity.

In a second mixing vessel the propanediol and carrageenan are mixed together to uniformity and are then admixed to the composition in the first mixing vessel. At his time further additives, medicaments, ingredients, etc. may be added and blended to uniformity.

The process can be carried out by a batch process, a continuous process or a combination of the two. The resulting lubricant composition may be scented or unscented, colored or colorless, flavored or flavorless, water-based, and/or may include humectants. The vaginal formula is pH balance with a range between 3.2 and 10.6, currently preferably 4.0. The viscosity range is between 200 to 30,000 cps, currently preferably 2,500 cps.

### EXAMPLE V

The following ingredients are provided in quantities to be admixed to produce a vaginal moisturizer/healing composition in the form of a gummy ring having the ingredients in the weight percent noted:

| | Weight % in |
|---|---|
| Ingredient | final composition |
| Carrageenan | 3.0 |
| Water | QS |
| Propanediol (Zemea TM) | 15.0 |
| Ganaden BC30 | 0.01% |
| Agave Inulin, spray dry | 0.50% |
| Menthol | 0.15% |
| Citric Acid | 0.13% |
| Potassium Sorbate | 0.45% |
| Sodium Benzoate | 0.05% |
| Sodium Hydroxymethylglycinate | 0.1% |
| Aloe | 0.1% |
| Vitamin E | 0.1% |

### Mixing Procedure

The water is metered into a first mixing vessel, and agitation is engaged.

The potassium sorbate, sodium benzoate, sodium hydroxymethylglycinate, citric acid, inulin, Ganaden BC30, evening primrose oil, menthol, niacin and L-arginine are added to the water an mixed to uniformity.

In a second mixing vessel the propanediol and carrageenan are mixed together to uniformity and are then admixed to the composition in the first mixing vessel to produce a preliminary gummy ring composition. At his time further additives, medicaments, ingredients, etc. may be added and blended to uniformity.

The preliminary gummy ring composition is poured into a doughnut-shaped mold. The mold is heated to evaporate moisture from the preliminary gummy ring composition until 10% water by weight remains in the composition. The resulting gummy ring is substantially solid, but is soft and pliable in the manner of a gum drop. The gummy ring can be inserted in a woman's vagina to dissolve gradually and moisturize and heal tissue in the vagina. The lubricant compositions and other compositions described herein are also administered in a woman's vagina.

The process can be carried out by a batch process, a continuous process or a combination of the two. The resulting lubricant composition may be scented or unscented, colored or colorless, flavored or flavorless, water-based, and/or may include humectants. The vaginal formula is pH balance with a range between 3.2 and 10.6, currently preferably 4.0. The viscosity range is between 200 to 30,000 cps, currently preferably 2,500 cps.

Having described our invention in such terms as to enable those skilled in the art to understand and practice it, and having described the presently preferred embodiments thereof, we claim:

1. A method to reduce sexual discomfort for a woman during sexual intercourse by vaginally introducing lubricant composition comprising
(a) carrageenan comprising 0 to 92% by weight iota carrageenan, 10% to 100% by weight lambda carrageenan; and, 0 to 18% by weight kappa carrageenan;
(b) water such that the ratio of carrageenan to water is in the range of 1:10 to 1:50;
(c) at least one prebiotic;
(d) a natural humectant with a peak tackiness force of less than 50 g and a work of adhesion of less than 0.500 GS.

2. The method of claim 1 wherein said composition includes at least one probiotic.

3. The method of claim 1 wherein said probiotic is Bacillus coagulan, said prebiotic is agave inulin, and said humectant is 1,3 propanediol.

4. A method to reduce sexual discomfort and improve sexual pleasure during intercourse by vaginally introducing a lubricant composition comprising
(a) a lubricant;
(b) a probiotic;
(c) a vasodilator;
(d) a sensate,
(e) a natural humectant with a peak tackiness force of less than 50 g and a work of adhesion of less than 0.500 GS.

5. The method of claim 4 wherein said lubricant is carrageenan, said probiotic is Bacillus coagulans, said vasodilator is selected from a group consisting of niacin and L-arginine, said topical stimulant is selected from a group consisting of menthol and evening primrose oil, and said natural humectantis 1,3 propanediol.
